(19) United States
(12) Patent Application Publication
(10) Pub. No.: US 2011/0177181 A1
(43) Pub. Date: Jul. 21, 2011

(54) Modified grape seed oils

(76) Inventors: Ralph Eric Leber, Prosser, WA (US); Daniel Perlman, Arlington, WA (US)

(21) Appl. No.: 12/927,970

(22) Filed: Nov. 29, 2010

Related U.S. Application Data
(63) Continuation of application No. 12/041,624, filed on Mar. 3, 2008, now abandoned.

(51) Int. Cl. A61K 36/87; A23D 9/00; A23L 1/24; A23L 1/39; A23D 7/00; A61Q 19/00; A61Q 19/10

(52) U.S. Cl. 424/766; 426/601; 426/605; 426/589

(57) ABSTRACT

Edible oils are described which are extracted from dried fermented grape seeds isolated from a fermented grape pomace that has undergone fermentation, e.g., primary fermentation during wine making. The edible oils contain distinguishing flavor and fragrance chemicals evidenced by organoleptic evaluation and chemical analysis, in which these chemicals are substantially lacking in grape seed oils that have been similarly extracted from grape seeds isolated from non-fermented grape pomace.

### RELATED APPLICATIONS

This application is a continuation of Leber and Perlman, application Ser. No. 12/041,624 which is incorporated herein by reference in its entirety.

### FIELD OF THE INVENTION

The present invention relates to edible oils extracted from dried grape seed obtained from fermented grape pomace.

### BACKGROUND OF THE INVENTION

The following discussion is provided solely to assist the understanding of the reader, and does not constitute an admission that any of the information discussed or references cited constitute prior art to the present invention.

In the process of winemaking, fresh grapes are crushed and allowed to ferment, either in the presence or absence of the crushed grape solids. During this primary fermentation, which often takes between one and two weeks, yeast converts most of the sugars in the grape juice into ethanol. Red wine is made from crushed red or black grapes that undergo fermentation together with the solids from the grapes including pulp residue, skins, seeds and some stems. Following fermentation, these solids are collectively referred to herein as “fermented pomace”. After the primary fermentation, the liquid is separated from the fermented pomace material and transferred to vessels for the secondary fermentation. Here, the remaining sugars are slowly converted into alcohol and the wine becomes clear as residual sediment or lees settle from the wine.

By contrast, white wine is usually made after having removed the solid skins, seeds, pulp residue and stems from the juice that has been pressed from white grapes. These grape solids from freshly pressed grapes are collectively referred to herein as “non-fermented pomace”. White wine (and rose wine) can also be made from must extracted from red grapes with little (or limited) contact with the grapes' skins.

Non-fermented grape pomace obtained from white wine production tends to be a pale, yellow-brown color and contains some residual sugars. Grape pomace has been extracted to yield natural red dye and food coloring, cream of tartar, and grape polyphenolic antioxidants. Grape seeds recovered from non-fermented pomace have, to a limited extent, been used to produce grape seed oil. On the other hand, fermented pomace from red wine production is a darker purple color and contains some alcohol as well as higher levels of tannins. Along with non-fermented pomace residues, fermented pomace has been either treated as waste requiring disposal or has been used as fodder, fertilizer or as a renewable energy source.

Grape seed oils are edible triglyceride-based oils. As indicated above, currently produced grape seed oils are extracted from seeds of non-fermented grape pomace (from grape juice and white wine production). Pomace from freshly pressed wine grapes that is commercially available is usually collected and pooled from multiple fields and vineyards. Therefore, commercial grape pomace usually contains the seeds from multiple varieties of grapes, and the resulting dried seeds as well as their expressed oils derive from multiple varieties of fresh (non-fermented) grapes.

There have been a small number of producers of grape seed oils that have acquired single varietal non-fermented grape seeds through producers of white wines and pressed them for oil. In fact, Applicant has prepared and tasted two such varietal grape seed oils extracted from the seeds of Chardonnay and White Riesling white wine grapes. These oils (further analyzed herein below) each have agreeable flavors not unlike the flavor of commercially available grape seed oils pressed from seeds of mixed varieties of non-fermented grapes.

### SUMMARY OF THE INVENTION

The present invention is directed to edible grape seed oils extracted from dried seeds obtained from fermented grape pomace, a source which has been previously regarded as only a waste product. Surprisingly, it was discovered that the oil from seeds obtained from the fermented grape pomace was modified as compared to oil from unfermented grape seeds, and that those differences imparted unique aroma and flavor properties to the oil. As a result, the present modified grape seed oils have surprising properties, e.g., allowing them to be matched with other food flavors in a complementary fashion.

Thus, a first aspect of the invention concerns a modified edible oil, which is or includes a modified edible grape seed oil extracted from dried fermented grape seeds. Such fermented grape seeds are usually isolated from a fermented grape pomace, e.g., a fermented grape pomace that has undergone primary fermentation. For example, the grape pomace may be fermented during wine making. The modified edible grape seed oil can contain distinguishing flavor and fragrance chemicals which may be evidenced by organoleptic evaluation and/or chemical analysis, where those chemicals are substantially lacking (or present to a significantly lesser extent) in grape seed oils that have been similarly extracted from grape seeds isolated from a non-fermented grape pomace, e.g., where the comparison is between oil from seeds in fermented grape pomace from red wine grapes (e.g., crushed grapes for winemaking) and oil from seeds from non-fermented grape pomace from white wine grapes (e.g., crushed for winemaking).

In certain embodiments, the modified grape seed oil is physically extracted (e.g., pressed, preferably cold pressed or alternatively pressed at very low temperature) from the dried fermented grape seeds without solvent extraction; the modified grape seed oil is obtained by a physical extraction followed by a solvent extraction (e.g., a non-polar and/or polar solvent extraction); the oil is purified following any of the types of extraction, e.g., by filtering and/or centrifugation.

In particular embodiments, the distinguishing flavor and fragrance chemicals are evidenced by GCMS analysis of ethanolic extracts obtained from the grape seed oils; the distinguishing flavor and fragrance chemicals include at least one, two, or three organic alcohols, e.g., isobutanol, isoamyl alcohol and/or phenylethyl alcohol; the distinguishing flavor and fragrance chemicals include at least 1, 2, 3, 4, 5, 6, 7, 8, 9, or 10 organic esters, e.g., ethyl propionate, ethyl butyrate, isoamyl acetate, ethyl caproate, hexyl acetate, ethyl caprylate, ethyl decylate, isoamyl caprylate, ethyl laurate, and/or ethyl myristate; the distinguishing flavor and fragrance chemicals comprise at least 1, 2, 3, 4, 5, or 6 organic acids, e.g., acetic acid, isovaleric acid, caproic acid, caprylic acid, decanoic acid, and/or lauric acid; the distinguishing flavor and fragrance chemicals include selections of each of the just specified organic alcohols, organic esters, and organic acids, e.g., any combination of at least 1, 2, or 3 of each of the specified chemical classes; the distinguishing flavor and fragrance chemicals are present at levels specified in Table 1 and/or in the Detailed Description herein.

In further particular embodiments, the fermented grape pomace is produced from at least one varietal grape from a following grape species: Vitis vinifera (European wine grape), Vitis labrusca (Concord), and Vitis rotundifolia (Muscadine); the fermented grape pomace is produced from a single varietal grape, e.g., of the species Vitis vinifera; the fermented grape pomace is produced from a combination of varietal grapes, e.g., of the species Vitis vinifera; the fermented grape pomace is produced from the Merlot or Cabernet Sauvignon varietal grape varieties of the species Vitis vinifera; the fermented grape pomace is produced from the grape species Vitis vinifera, including at least one varietal grape from the following: Barbarossa, Barbera, Cabernet Franc, Cabernet Sauvignon, Castiglione, Charbono, Corvina, Dolcetto, Dornfelder, Freisa, Gamay, Graciano, Gropello, Grignolino, Lambrusco, Malbec, Malvasia, Mandolari, Merlot, Millot, Mission, Molinara, Montepulciano, Negrette, Nerello, Petit Syrah, Petit Verdot, Pinot Noir, Pinotage, Poulsard, Rodinella, Rotberger, Rufete, Sagrantino, Sangiovese, Shiraz, Syrah, St. Laurent, Tarrango, Terret Noir, Touriga, Troia, Vranac, and Zinfandel.

In certain embodiments, the residual moisture content of grape seeds prior to oil extraction is between approximately 4% and 10% by weight, e.g., approximately 5, 6, 7, or 8% by weight, of the residual moisture content is in a range of approximately 4-6, 5-7, 6-8, 7-9, 8-10, or 5-9% by weight, where the term approximately indicates that conventional rounding of the precise value to the nearest integer will result in the specified integer value.

Also in certain embodiments, the modified edible oil as specified above is used as an ingredient in a personal care or cosmetic product, e.g., massage oils, skin care oils, skin cleansers, skin moisturizers, skin serums, skin creams, body washes, body butters, body muds, body scrubs, bath gels, bath lotions, sun-blocking oils and sun lotions; the modified edible oil as specified above is used for cooking another food, e.g., by stir frying or sautéing; the modified edible oil as specified above is used as an ingredient in another prepared food product, e.g., salad dressings, dips, marinades, rubs, salsas, cooking sauces, and pasta sauces.

In further embodiments, the present modified edible oil is a blend of a modified edible grape seed oil as specified above with another edible oil, e.g., a grape seed oil from non-fermented grape seeds, another edible fruit seed oil, e.g., raspberry seed oil, cranberry seed oil, and/or blueberry seed oil, and/or a non-fruit edible vegetable oil, e.g., olive oil, soybean oil, canola oil, corn oil, peanut oil, flax seed oil, and/or palm oil.

In still further embodiments, the modified edible oil contains at least one non-grape flavor property, e.g., smoke, citrus, herbal, savory, and/or pepper flavors, preferably with the flavors derived from the actual source material instead of using synthetic flavoring.

Thus, a related aspect of the invention concerns an oil blend which includes a modified edible oil as specified for the aspect above combined with a second edible triglyceride-based oil. Preferably the distinguishing flavor and fragrance chemicals as described above or otherwise described herein for the present invention contribute to the flavor and fragrance of the oil blend.

In particular embodiments, the second edible oil is another edible fruit seed oil, e.g., raspberry seed oil, cranberry seed oil, and/or blueberry seed oil, and/or a non-fruit edible vegetable oil, e.g., olive oil, soybean oil, canola oil, corn oil, peanut oil, flax seed oil, and/or palm oil.

Another related aspect of the invention concerns a prepared food which includes an edible oil as specified for either of the preceding aspects. Preferably the oil includes distinguishing flavor and fragrance chemicals from fermented grape seeds which contribute to the flavor and fragrance of the food product; the prepared food is a salad dressing, dip, marinade, rub, salsa, cooking sauce, or pasta sauce.

Further related aspects concern methods for preparing a modified grape seed oil as specified in an above aspect, by extracting oil from fermented grape seeds in a manner which retains distinguishing flavor and fragrance chemicals, e.g., using cold pressing of dried fermented grape seeds to extract modified grape seed oil.

Likewise, in yet another related aspect, the invention concerns methods for preparing a prepared food product which includes an edible oil of this invention by using the present edible oil as at least part of the oil in an oil-containing food product. The oil can usually be incorporated by techniques normally used for the particular food product, e.g., mixing, high speed blending, emulsifying, and the like. Of course, the preparation for the food product may include additional techniques, e.g., frying, baking, and the like.

Still further, in view of the fact that grape seeds are recognized as having beneficial health properties, the invention provides nutraceuticals which include a modified grape seed oil as described herein. In certain embodiments, the oil is enriched in one or more compounds extracted from grape seeds, for example, antioxidants and/or plant sterols (e.g., obtained from solvent extraction of grape seeds such as after oil has been pressed from the seeds).

Additional embodiments will be apparent from the Detailed Description and from the claims.

### DETAILED DESCRIPTION OF THE PREFERRED EMBODIMENTS

To assist the understanding of the reader, in discussing the present invention and in the claims, the following terms are applicable and have the indicated meanings.

### Definitions

The term “edible oil” within the context of the present invention refers to a triglyceride-based edible grape seed oil obtained from fermented grape seeds.

The term “fermented” refers to a process of primary yeast-based fermentation that takes place over a period of time, usually ranging from a few days to approximately two weeks. While red or white grapes may be fermented in producing a fermented grape pomace, red grapes are preferred because greater flavor is generally developed in the grape seeds.

The term “fermented grape pomace” refers to the mixed solids in the crushed grape fermentation mixture, typically including grape skins, seeds and stems. These solids accompany the fermentation of the liquid portion of the grapes.

The term “fermented grape seeds” refers to grape seeds that have been exposed to fermentation, usually as part of a grape pomace. Usually the grape pomace is dried, and the seeds separated from the other dried pomace material to provide dried fermented grape seeds. The extent of grape seed drying is important for maximizing extraction of the oil and minimizing production of water-oil emulsions during the pressing operation.

The term “physical extraction” refers to the method of recovering the edible oil from the seeds, and refers to the use of mechanical means to force the oil from the seeds, e.g., “cold-pressing,” rather than the use of caustic (alkaline) and/or organic solvent means of extraction such as hexane extraction often used with commercial production of soybean oil, some grape seed oils, canola, peanut oil and the like.

The term “distinguishing flavor and fragrance chemicals” refers to chemical compounds produced during fermentation (e.g., primary fermentation), and found in grape seed oils from fermented grape seeds at levels substantially higher, e.g., at least four-fold and often ten-fold or more higher, than in the oils from non-fermented grape seeds. The differences in flavors and fragrances can be evidenced by organoleptic evaluation and/or chemical analysis of the ethanolic extracts of the different oils. Most often, the distinguishing flavors and fragrances of grape seed oils that are extracted from fermented grape seeds are undetectable in oils from non-fermented grape seeds.

The term “chemical analysis” used in the present invention to distinguish flavor and fragrance chemicals present in grape seed oils from fermented versus non-fermented grape seeds refers to the coupled use of gas chromatography linked to mass spectroscopy (GCMS). GCMS is extensively used to analyze perfume and flavor compositions by manufacturers of these products. The GCMS analyses provided herein were performed on ethanolic extracts obtained by extracting 15 g of grape seed oil with 1 g of aqueous ethanol (90% by weight ethanol, 10% by weight water). Oils from fermented Cabernet Sauvignon and Merlot varietal grape seeds were compared with oils from non-fermented Chardonnay and White Riesling varietal grape seeds.

The term “organoleptic” as used herein refers to sensory tasting and smelling evaluation methods used for differentiating or distinguishing flavors and fragrances (representing chemical differences) in grape seed oils, and the corresponding taste and aroma properties. Before the decision was made to carry out GCMS analysis of the four grape seed oils that Applicant had pressed, a consensus was reached among six wine tasters that the grape seed oils from fermented grape seeds, when compared with those from non-fermented seeds, contained a considerably richer and more intense bouquet of fragrances and flavors.

The term “cold-pressing” as used herein refers to the physical recovery, i.e., expression, and purification of edible oils from grape seeds using a high pressure mechanical device or press (without the use of organic solvent extraction) to force the oil from the seeds. Generally the cold-pressing process is carried out such that the temperature of the seeds and expressed oil does not exceed about 175 degrees F. and preferably does not exceed about 120 degrees F. The oil-bearing grape seeds are dried to a certain level of residual moisture. Preferably, the grape seeds are dried to a residual moisture content of between 4% and 10% by weight, and more preferably to a moisture level of approximately 7% of the grape seed weight. Thereafter, the seeds are subjected to mechanical pressing before filtering to obtain pure oil.

The term “solvent extraction” refers to the use of solvents, usually organic solvents, to recover edible oils. Most often such organic solvent extraction employs hexane. Solvent extraction commonly allows greater total recovery of edible oils. Preferably it is not used, or not used as the primary oil extraction method, for preparing the present oils because it can result in the loss of desirable volatile flavors and fragrances. In addition, a commonly used oil extraction solvent, hexane, is a toxic solvent and is environmentally hazardous. Nonetheless, solvent extraction may be used, e.g., to increase the oil recovery and/or when the solvent selected does not unacceptably reduce the resulting concentrations of desired flavor and/or fragrance compounds.

The terms “filtration” and “centrifugation” have their conventional meanings, and, in the present context, are physical methods which may be used to remove particulate materials, thereby further purifying grape seed oil, e.g., following cold-pressing.

### General Description

As discussed in the Summary above, this invention concerns modified grape seed oils extracted from the dried seeds of fermented grapes, typically fermented viniferous (wine-making) grapes. More specifically, after the primary fermentation of wine grapes is complete, the grape pomace, that contains grape skins, stems and seeds, is collected and dried, and the seeds are physically separated from the remainder of the pomace and processed to obtain the seed oil. The fermentation results in modification of the oil obtained from the grape seeds. Extraction of the oil from the grape seeds thus provides an edible modified grape seed oil basic to the present invention. Advantageously the oil is extracted using mechanical extraction, e.g., cold-pressing. For cold-pressing of the modified grape seed oil, the residual moisture level in the seeds should preferably be reduced to be between 4% and 10% by weight, and more preferably approximately 7% by weight.

Because of the general belief that fermented grape pomace is a waste by-product without further application to preparation of edible products for humans, and in view of the ready availability of grape seeds from freshly crushed wine grapes, the production of distinctive modified grape seed oil from the seeds of fermented grape pomace is unexpected, and such oils have been unavailable. To the contrary, fermented grape pomaces are typically returned to the vineyard, used for animal feed (fodder), discarded in landfills, or burned for generating energy.

In the process leading to the production of modified grape seed oil from fermented grape seeds, one or more types of varietal grapes are crushed and fermented, e.g., according to standard vinifying methods. As discussed above, the fermented pomace from the primary fermentation is collected and dried, and the dried grape seeds are physically separated from the other pomace components. For practical reasons, it is usually preferable to dry the pomace and then separate the seeds, but separating the seeds and then drying the separated seeds can also be done. The seeds are preferably cold-pressed to release their edible oils. While cold-pressed oils cannot exceed a temperature of 175 degrees F., cold-pressed fermented and non-fermented oils prepared by the Applicant preferably do not exceed 120 degrees F. Such physical extraction of the oil without the use of organic solvents is useful for preserving natural flavors and fragrances in the fermented grape seed oil. Optionally, the pressed oils can be filtered and/or centrifuged by conventional means to remove small amounts of fine solid particulates that may be considered undesirable.

Also optionally, solvent extraction may be used after pressing to increase the yield of oil. With careful selection of solvent in view of the characteristic compounds contributing to the organoleptic properties for the particular variety or varieties being processed, solvent extraction may be used as the sole or primary extraction method. For example, a solvent may be chosen which allows desired constituent compounds in the oil to be preserved in the oil and not lost in the solvent. Such extraction can be used to increase the content of sterols and stanols (collectively phytosterols) in the resulting oil. Such increased phytosterol content can be advantageous, e.g., for reducing the uptake of dietary cholesterol.

An additional optional extraction from the grape seeds involves the use of a polar solvent, for example, a combination of water and ethanol. Such a solvent can extract polar species from the grape seeds, such as a variety of antioxidant compounds.

Polar and non-polar solvent extractions can thus provide enrichment in beneficial phytochemicals such as plant sterols and stanols and related compounds (collectively phytosterols), as well as polar antioxidant species.

The modified oils from fermented grape seeds (and particularly cold-pressed oils) have been found to possess remarkable flavor and fragrance profiles which differ from grape seed oils extracted from the seeds of non-fermented varietal wine grapes. While many water and alcohol-soluble flavors including many ester-based compounds are known to develop from fermentation enzymes in a fermenting wine, Applicant hypothesizes that enzymatic esterification extends into the interior of the grape seed where oils are found. The basis for this hypothesis and proposed esterase enzymatic source of flavors and fragrances in fermented grape seeds is based upon finding many ethyl ester derivatives of fatty acids in the gas chromatographic mass spec (GCMS) analytical profile of the grape seed oil from fermented pomace grape seeds (see below) which are essentially absent from the oils from the non-fermented pomace grape seeds. Whether this is a result of exogenous or endogenous esterase enzymatic activity is unknown at present. However the agreeable organoleptic result, i.e., improved flavor and fragrance in grape seed oils from fermented grape seeds, is surprising and has commercial value.

Thus, in accordance with the term “distinguishing flavor and fragrance chemicals”, it was discovered that a large number of organic chemical species that are grouped within a small number of organic chemical classes are responsible for the flavor and fragrance differences between modified grape seed oils from fermented grape seeds and grape seed oils from non-fermented grape seeds. These differentiating chemical classes include the organic alcohols, organic esters, and organic acids within which the organic ester class contains the greatest species diversity (see below).

With regard to specific chemicals within the differentiating chemical classes, identified differentiating organic alcohols include isobutanol, isoamyl alcohol, C6 and/or C7 alcohols (hexanol, heptanol) and phenylethyl alcohol. Identified differentiating organic esters include ethyl propionate, ethyl butyrate, isoamyl acetate, ethyl caproate, hexyl acetate, ethyl caprylate, ethyl decylate, isoamyl caprylate, ethyl laurate, and ethyl myristate. Identified differentiating organic acids include isovaleric acid, caproic acid, caprylic acid, decanoic acid and lauric acid.

Therefore, the present invention concerns grape seed oils (e.g., from fermented grape seeds or supplemented with at least one and preferably a plurality of the specified chemical species at levels elevated significantly above those of the corresponding unfermented grape seed oil, e.g., elevated at least 2, 3, 4, 5, 7, 10, 20, 30, 50, or 100-fold compared to the corresponding unfermented grape seed oil (such as a particular varietal grape seed oil). For example, such oils may include (with exemplary levels specified in percent scaled to the total of identified extracted compounds as was done for identified compounds listed in Table 1 below (that is, the percentages of the particular compounds are the percentages that each compound constitutes of the total identified extracted compounds for the respective oil sources when extracted from the oil using the specified solvent)):
isobutanol (e.g., at a level of at least 0.30, 0.40, 0.50, 0.60, 0.70, or 0.80%; or in a range of 0.30-1.00, 0.50-0.90, or 0.60-0.90%);
iso amyl alcohol (e.g., at a level of at least 0.5, 1.0, 2.0, 3.0, 5.0, 7.8, 10.0, or 15.0%, or in a range of 0.5-3.0, 2.0-5.0, 3.0-10.0, 5.0-10.0, 7.0-15.0, 3.0-20.0, 7.0-20.0, or 10.0-20.0%);
isoamyl acetate (e.g., at levels of at least 0.03, 0.05, 0.07, 0.10, 0.12, 0.15, 0.20, 0.30, or 0.50%, or in a range of 0.03-0.50, 0.03-0.30, 0.03-0.20, 0.05-0.50, 0.05-0.30, 0.05-0.20, 0.10-0.50, 0.10-0.30, or 0.10-0.20);
caproic acid (e.g., at a level of at least 0.15, 0.20, 0.25, 0.30, 0.35, or 0.40%, or in a range of 0.15-0.30, 0.15-0.40, 0.20-0.35, 0.25-0.35, or 0.25-0.50%);
ethyl caproate (e.g., at a level of at least 0.15, 0.20, 0.25, 0.30, 0.50, 0.70, 1.00, 1.2, or 1.5%, or in a range of 0.15-0.50, 0.20-0.70, 0.25-2.00, 0.30-1.00, 0.50-2.00, or 1.00-2.00%);
phenyl ethyl alcohol (e.g., at a level of at least 0.20, 0.50, 0.70, 1.00, 1.20, 1.50, 1.70, 2.00, 2.50, or 3.00%, or in a range of 0.20-0.70, 0.50-1.00, 1.00-2.00, 1.00-3.00, 1.00-4.00, 1.50-4.00, 1.50-3.50, 1.50-2.50, 2.00-4.00, or 2.00-3.50%);
caprylic acid (e.g., at a level of at least 0.10, 0.20, 0.30, 0.40, 0.50, 0.60, or 0.70%, or in a range of 0.10-0.50, 0.20-0.80, 0.30-0.80, 0.40-1.00, 0.50-1.00, 0.50-0.80, or 0.50-1.20%);
ethyl caprylate (e.g., at a level of at least 0.30, 0.50, 0.70, 1.00, 1.20, 1.50, 1.70, 2.00, 2.50, or 3.00%, or in a range of 0.10-0.50, 0.30-1.00, 0.50-1.50, 0.50-2.00, 0.50-3.00, 1.00-2.00, 1.00-3.00, 1.00-3.50, 1.00-4.00, 2.00-4.00, 2.00-3.50, or 2.00-3.00%);
decanoic acid (e.g., at a level of at least 0.20, 0.30, 0.40, 0.50, 0.70, 1.00, 1.20, 1.50, or 1.70%, or in a range of 0.20-0.50, 0.50-1.00, 0.70-1.00, 0.70-2.00, 0.80-1.20, 0.80-1.70, 0.80-2.00, 1.00-1.50, 1.00-2.00, or 1.50-2.00%);
ethyl decylate (e.g., at a level of at least 0.20, 0.30, 0.40, 0.50, 0.70, 1.00, 1.20, 1.50, 1.70, 2.00, 2.20, 2.50, 1.70, 3.00, or 3.50, or in a range of 0.20-0.50, 0.50-0.70, 0.50-1.00, 0.50-1.50, 1.00-1.50, 1.00-2.00, 1.00-3.00, 1.00-4.00, 1.50-3.00, or 1.50-4.00);
lauric acid (e.g., at a level of at least 0.20, 0.30, 0.40, 0.50, 0.70, 1.00, 1.20, 1.50, or 1.70%, or in a range of 0.20-0.50, 0.50-1.00, 0.70-1.00, 0.70-1.50, 0.70-2.00, 0.80-1.20, 0.80-1.70, 0.80-2.00, 1.00-1.50, 1.00-2.00, or 1.50-2.00%); and/or
ethyl laurate (e.g., at a level of at least 0.10, 0.20, 0.30, 0.40, 0.50, or 0.70%, or in a range of 0.10-0.30, 0.3-0.50, 0.30-0.70, 0.30-1.00, 0.40-0.70, 0.40-1.00, or 0.50-1.00%).

As seen from Table 1, differentiating chemicals (i.e., distinguishing flavor and fragrance chemicals) commonly occur in combination, thereby providing more complex organoleptic properties. For example, a present oil may include iso butanol and iso amyl alcohol (e.g., at 0.50-1.00 and 3.0-20.0% respectively or any other combination of the percentage compositions specified above for these components). Similarly, the oil may include iso amyl alcohol and iso amyl acetate (e.g., at 3.0-20.0 and 0.03-0.50% respectively or any other combination of the percentage compositions specified above for these components). Likewise, the oil may include caproic acid and ethyl caproate (e.g., at 0.15-0.50 and 0.20-2.0% respectively or any other combination of percentage compositions specified above for these components). The oil may include caprylic acid and ethyl caprylate (e.g., at 0.30-1.00 and 0.50-4.00% respectively or any other combination of percentage compositions specified above for these components). The oil may include decanoic acid and ethyl decylate (e.g., at 0.3-2.5 and 0.30-3.00% respectively or any other combination of the percentage compositions specified above for these components). The oil may include lauric acid and ethyl laurate (e.g., at 0.2-2.5 and 0.1-1.0% respectively or any other combination of percentage compositions specified above for these components).

In further combinations, a present oil may include isoamyl acetate and ethyl caproate; or isoamyl acetate, ethyl caproate, and ethyl caprylate; or isoamyl acetate, ethyl caproate, ethyl caprylate, and ethyl laurate; or ethyl caproate and ethyl caprylate, or ethyl caproate, ethyl caprylate, and ethyl laurate; or ethyl caprylate and ethyl laurate; in particular combinations the percentages of each of the specified components is each combination of the respective values and/or ranges specified above.

In still further combinations, a present oil may include iso amyl alcohol and ethyl caproate; or iso amyl alcohol, isoamyl acetate, and ethyl caproate; or isoamyl alcohol, isoamyl acetate, ethyl caproate, and ethyl caprylate; or iso amyl alcohol, isoamyl acetate, ethyl caproate, ethyl caprylate, and ethyl laurate; or iso amyl alcohol, ethyl caproate and ethyl caprylate, or iso amyl alcohol, ethyl caproate, ethyl caprylate, and ethyl laurate; or iso amyl alcohol, ethyl caprylate and ethyl laurate; or iso amyl alcohol and ethyl laurate; in particular combinations the percentages of each of the specified components is each combination of the respective values and/or ranges specified above.

As indicated, the percentages and percent ranges specified above for identified extracted compounds correspond to percentages normalized such that the total for all identified compounds for each oil is 100%. Corresponding percentages and ranges may instead be expressed in terms of the percentage that each of the compounds represents of the total extracted compounds, that is, percentages expressed in the manner of Table 2.

While wine grapes of the genus and species Vitis vinifera are generally the preferred grapes for producing the modified grape seed oils described herein, the edible oil can be obtained from any single edible grape species (or variety within a single species) including the species Vitis vinifera (European wine grape), Vitis labrusca (Concord), and Vitis rotundifolia (Muscadine) or, in fact, mixture of two or more edible species and/or varieties.

For example, considering the varietal grapes, a fermented grape pomace can be produced and used as the source of fermented grape seeds, and of the resulting modified oil, such as from the Merlot varietal grape variety of the species Vitis vinifera, and/or from Cabernet varietal grapes of the species Vitis vinifera.

As further examples, the fermented grape pomace, its seeds and the resulting modified edible oil can be produced from the grape species Vitis vinifera, in which at least one varietal grape of this species is selected from the group of varietals consisting of Barbarossa, Barbera, Cabernet Franc, Cabernet Sauvignon, Castiglione, Charbono, Corvina, Dolcetto, Dornfelder, Freisa, Gamay, Graciano, Gropello, Grignolino, Lambrusco, Malbec, Malvasia, Mandolari, Merlot, Millot, Mission, Molinara, Montepulciano, Negrette, Nerello, Petit Syrah, Petit Verdot, Pinot Noir, Pinotage, Poulsard, Rodinella, Rotberger, Rufete, Sagrantino, Sangiovese, Shiraz, Syrah, St. Laurent, Tarrango, Terret Noir, Touriga, Troia, Vranac, and Zinfandel.

Applicant has prepared, tasted, and chemically analyzed cold-pressed varietal grape seed oils isolated from seeds separated from both non-fermented and fermented grape pomaces. These include non-fermented Chardonnay and White Riesling varietal grape pomaces and also fermented Merlot and Cabernet Sauvignon varietal grape pomaces from grapes grown in the State of Washington. Results of the chemical analysis are shown in Table 1 provided below.

### Blends of Modified Grape Seed Oils

In addition to modified grape seed oils derived from a single varietal grape, useful oil blends may be made, which may be of any of a number of different types.

In one type of blend, modified grape seed oils from two or more varieties of fermented grape pomace are blended. Such blending may be performed after extraction of the oil, and/or oil may be extracted from a mixture of two or more different fermented grape pomaces. Blending after extraction of the modified varietal oils can be advantageous, e.g., because it allows greater control of the organoleptic properties of the resulting blended modified grape seed oil.

Similarly, a modified grape seed oil or oil blend may be blended with one or more grape seed oils extracted from non-fermented grape seeds, such as the Chardonnay and White Riesling varieties.

The present modified varietal grape seed oils and blends can also be blended with other types of oils (non-grape seed oil), e.g., to reduce the flavor and/or aroma intensity, to impart desired organoleptic properties to the modified grape seed oil and/or to the other type(s) of oil, and/or as a lower cost bulking oil. For example, a modified grape seed oil may be blended with olive, soy, canola, corn, peanut, palm, or flax seed oil.

A particularly desirable type of blend uses a modified grape seed oil blended with another type of edible fruit seed oil, e.g., raspberry seed oil, cranberry seed oil, or blueberry seed oil. The proportions used can be varied as desired, e.g., to provide the desired combination of organoleptic properties. Indeed, Applicants have found that combinations of grape seed oil and other edible fruit seed oils can be advantageously made using either modified grape seed oils or grape seed oils from non-fermented grape seeds.

### Additional Modifications of Organoleptic Properties

In addition to the creation of oil blends, the present modified oils and oil blends, as well as grape seed oils from non-fermented grape seeds can be flavor modified in a number of different ways, which in some cases can produce unexpectedly desirable flavor combinations, e.g., in which the flavor of the grape seed oil and an added flavor are particularly complementary.

A unique example is the use of smoke flavoring, e.g., obtained by bubbling smoke through modified grape seed oil. Combinations found to be quite desirable are produced by infusing smoke flavor (e.g., bubbling smoke from apple wood or from viniferous grape vines, e.g., which have been harvested and made into smoking chips) through modified grape seed oil from Merlot seeds or Chardonnay seeds.

An additional useful method for instilling flavor components in the present modified grape seed oils is by co-pressing one or more additional flavor/aroma source materials with the grape seeds. For example, such source materials may be other oil seeds, such as the fruit seeds mentioned herein (e.g., raspberry, cranberry, and blueberry). Likewise, the source material may be a material such as citrus peel (e.g., lemon, lime, or orange peel, especially the outer rind portion, often referred to as zest). A variety of other source materials may also be used.

In addition, further steps may be taken which provide flavor modifications. For example, additional and/or non-standard fermentations with grape seeds (usually as part of grape pomace) may be carried out and alter the organoleptic properties of the resulting oil. For examples, strains of Saccharomyces cervisiae may be used which produce a different spectrum of alcohols (rather than just ethanol) as co-metabolites. This can result in the production of additional types of esters, adding more complex and interesting flavor and aroma profiles for the oils.

Likewise, additional esters may be produced by basic (e.g., using NaOH) or acidic catalysis using particular alcohols to form the corresponding esters. The profile of the resulting ester products in the oil can be adjusted as desired to provide an oil with selected organoleptic properties. Conditions for carrying out such reactions are well-known or can be readily determined.

Similarly, appropriate enzymes (e.g., esterases, reductases, and other such enzymes) can be used with the oil and, if needed, other reactants, to produce compounds which provide interesting and/or selected flavor and/or aroma profiles for the oil (e.g., esters, ketones, aldehydes, and/or alcohols).

In some cases, the additional modification is carried out directly on the bulk oil (or on the seeds or pomace before extraction). However, compounds resulting from any such processes as mentioned above and/or obtained from other sources may be blended with the oil to provide a further modified oil or blend. For example, a chemical profile may be determined, and a base oil may be further flavor modified be addition of selected compounds to produce a further modified oil matching that chemical profile.

Yet another approach is to use oil extraction at very low temperatures. As indicated above, preferably the present oils are cold pressed (e.g., at 120 degrees F. or lower, usually down to about normal room temperature). Pressing at such temperatures reduces the degradation of the oil and formation of undesirable compounds such as peroxides and free fatty acids. As a result, the pressing can be carried out at very low temperatures, and/or may be conducted in the presence of a less reactive gas environment than normal air. For example, the temperature of the seeds may be lowered, e.g., to between 0 and 50 degrees F. This can be accomplished using various cooling methods, such as using solid carbon dioxide and/or liquid nitrogen to provide cold gases which may be used to lower the grape seed temperature. In this case, the coolant gas(es) can also be used to displace the air (with its oxygen). The lower temperature can thus improve the nutritional and/or organoleptic properties of the resulting extracted oil.

In view of the discovery that the present modified grape seed oils possess distinctive chemical profiles which provide desirable organoleptic properties, other types of oils (e.g., non-fermented grape seed oil or non-grape seed oil) can be modified, e.g., using methods as described above, to create a chemical profile which provides desirable organoleptic properties. For example, particular chemical profiles can be correlated with particular organoleptic properties. In order to at least partially provide those properties using a different base oil, the different base oil can be modified to at least approximate the desired profile. The different base oil may, for example, be any of the common oils mentioned herein. Of course, a skilled person will recognize that the different fatty acid composition of the different base oils will usually cause some differences in organoleptic properties, even if the principal aromatic and flavor compounds are substantially matched.

### Additional Edible Products Containing Modified Grape Seed Oil

In many cases, the present edible oils are used in other food products or as nutraceuticals. Indeed, the present modified grape seed oils may be used in essentially any application for which other vegetable oils are used, preferably with consideration given to flavor compatibility and tolerance to heating. Modified grape seed oil may be used instead of all or part of the oils in food products such as salad dressings, dips, rubs, and marinades, as well as in cooking other foods, e.g., sautéing of vegetables and baking.

For different uses, delivery of the oil may be varied as appropriate for the application. Due to its tolerance to substantial heating, the present modified grape seed oils may be used in many different ways for cooking, e.g., similar to common vegetables oils such as canola oil. Additionally, due to the flavors and aromas of these modified grape seed oils, they may be used in a manner similar to olive oil, e.g., in salads and the like. Especially in view of the substantial or even intense flavors of some of the present modified oils, efficient delivery methods can be advantageous. Examples include spraying, e.g., using pump or pressurized gas sprays. Such spraying can be particularly beneficial for grilling or frying, or any other application where a thin coating is desired.

### Example 1

### GCMS Analysis of Ethanolic Extracts of Varietal Grape Seed Oils

Filtered cold-pressed varietal grape seed oils were prepared from varietal grape seeds recovered from either fermented or non-fermented grape pomaces, in which the grape seeds had been dried to a residual moisture content of 7% by weight prior to pressing. The non-fermented grape seeds were obtained from freshly pressed Chardonnay and White Riesling grape pomaces, while the fermented grape seeds were obtained from fermented grape pomaces that were recovered following completion of primary fermentation during the first stage of Merlot and Cabernet Sauvignon wine making. Following drying of these pomaces and recovery and cold-pressing of the dried grape seeds, the grape seed oils were evaluated by their flavor profiles as well as by chemical analysis.

Organoleptic characterizations (particularly flavor profiles), provided the following descriptions for the four oils:

(a) White Riesling—a mildly flavored oil having a fresh grassy flavor with a hint of citrus.

(b) Chardonnay—a smooth-tasting creamy oil having a buttery roundness.

(c) Merlot—a full-bodied oil having a robust and complex flavor with a Scotch-like finish.

(d) Cabernet Sauvignon—a full-bodied oil having distinctive fruit notes with a smooth honey-like finish.

For chemical analysis of the oils (coupled gas chromatography-mass spectroscopy), 15 g samples of the grape seed oils were extracted with approximately 1.0 g of 90% ethanol: 10% water by weight. These ethanolic extracts were centrifuged to eliminate any particulate material and shipped to the analytical laboratory of a perfume manufacturer (Belmay, Inc., Yonkers, N.Y.) for GCMS analysis. This company provided GCMS analytical data in which the identified flavor and fragrance chemicals contained in each ethanolic extract from each grape seed oil were normalized to a total of 100% to allow comparison of the chemical species in these varietal oils. Table 1 and 2 provide this comparison among the flavor and fragrance chemicals in grape seed oils from fermented (Cabernet Sauvignon and Merlot) and non-fermented (Chardonnay and Riesling) grape seeds. Table 1 shows the relative percentages of identified extracted compounds with the total identified extracted compounds scaled to 100%. Table 2 shows the concentrations of the extracted compounds as a percentage of all extracted compounds, including both identified and non-identified compounds. From an examination of Table 1 and Table 2 it is apparent that the fermented Cabernet Sauvignon and Merlot oils contain a wealth of flavor and fragrance chemicals that are absent in the un-fermented Chardonnay and White Riesling oils.

In the fermented grape seed oils it is evident that a number of ethyl ester derivatives of fatty acids are formed whose natural carbon backbone lengths (without counting the two ethyl ester carbons) differ from one another by 2 carbons (C2). For example, reading from higher to lower molecular weights, the analysis showed that ethyl derivatives of stearate (C18), linoleate (C18), palmitate (C16), myristate (C14), laurate (C12), decylate (C10), caprylate (C8), caproate (C6), butyrate (C4) and acetate (C2) were formed, while odd-numbered carbon chain esters were not found to be present. This is strong presumptive evidence that the esters are derived from natural fatty acids. While there are a few esters that may derive from other sources (e.g., ethyl propionate and isoamyl acetate), these seem to be the exceptions.

The carboxylic acid-containing molecules that were found to be present in the fermented grape seeds and their oils but either minimally, or not present in non-fermented grape seeds (and that further differentiate the oils) include isovaleric acid, caproic acid, caprylic acid, decanoic acid and lauric acid. With the exception of isovaleric acid, these molecules contain even numbers of carbon atoms, again suggesting their origin in triglycerides and their cleavage as free fatty acids.

The alcohol group-containing molecules that were found in fermented grape seeds and their oils but either minimally, or not present in non-fermented grape seeds include isobutanol, isoamyl alcohol, at least two long-chain primary alcohols including hexanol (C6) and heptanol (C7), and phenylethyl alcohol. While alcoholic fermentation is suggested as their origin, there is no more specific mechanism suggested.

With regard to the enhanced organoleptic profile of the Cabernet Sauvignon and Merlot oils from fermented grape seeds (compared to Chardonnay and White Riesling), the largest distinguishing group of flavor and fragrance molecules are the esters. These have been identified above, and possess a variety of fruity notes such as pineapple, pear, apple, banana, wine, floral notes and the like. With regard to the smaller distinguishing group of carboxylic acid-containing molecular species identified in the Cabernet Sauvignon and Merlot oils, these correspond to free fatty acids and provide a creamy to somewhat rancid milk flavor that is sufficiently mild so as to minimally contribute to the overall fragrance and flavor of the oils. Regarding the additional and relatively small distinguishing group of alcohols identified in the Cabernet Sauvignon and Merlot grape seed oils, these include phenylethyl alcohol that provides a floral rose flavor and fragrance. The C6 and C7 long-chain primary alcohols provide fruity flavors, and isobutanol and isoamyl alcohol providing a fruity-winey flavor. While an excessive level of these compounds, also known as fusel alcohols, would be disagreeable, the amounts described herein add flavor complexity to the oils.

TABLE 1

| | Normalized to 100% | | | |
|---|---|---|---|---|
| | CABERNET | MERLOT | CHARDONNAY | W. RIESLING |
| iso Propyl Alcohol | | | | |
| Ethyl Acetate | 0.61% | | | |
| Acetic Acid | | 0.07% | | 0.10% |
| iso Butanol | 0.87% | 0.65% | | |
| Ethyl Propionate | 0.17% | | | |
| Acetoin (3-Hydroxy-2-Butanone) | | 0.08% | | |
| iso Amyl Alcohol | 18.18% | 8.26% | 0.01% | |
| Ethyl Butyrate | 0.28% | | | |
| iso Valeric Acid | 0.07% | | | |
| Aldehyde C-6 | | | 0.20% | 1.00% |
| Octene | | | | 0.53% |
| Octane | | | 0.73% | 2.66% |
| Leaf Alcohol (cis-3-Hexenol) | 0.01% | | | |
| Alcohol C-6 | 1.67% | | 0.09% | 0.15% |
| iso Amyl Acetate | 0.16% | 0.05% | | |
| Aldehyde C-7 | 0.03% | | 0.15% | 0.19% |
| Alcohol C-7 | 0.00% | 2.59% | | |
| Caproic Acid | 0.25% | 0.31% | | 0.06% |
| 1-Octen-3-ol | 0.13% | | 0.09% | 0.54% |
| Ethyl Caproate | 1.90% | 0.31% | 0.05% | |
| Valeraldehyde Diethyl Acetal | | 0.02% | | |
| Hexyl Acetate | 0.14% | | | |
| Phenyl iso Butyrate | 0.00% | | | 0.18% |
| Phenyl Acetaldehyde | 0.02% | | | |
| Alcohol C-8 | 0.07% | | | |
| Ethyl Heptoate | 0.01% | | | |
| Phenyl Ethyl Alcohol | 3.21% | 2.15% | 0.04% | |
| Diethyl Succinate | | 0.03% | | |
| Caprylic Acid | 0.73% | 0.51% | | |
| Ethyl Caprylate | 3.39% | 2.28% | 0.02% | 0.00% |
| trans-2,trans-4-Decadienal | 2.20% | 1.46% | 2.43% | 8.41% |
| Decanoic Acid | 1.75% | 0.93% | | |
| Ethyl Decylate | 2.04% | 1.48% | | |
| iso Amyl Caprylate | 0.08% | | | |
| Lauric Acid | 1.49% | 0.78% | | |
| Ethyl Laurate | 0.40% | 0.36% | | |
| cis alpha Farnesol | 0.02% | | | |
| Benzyl Benzoate | | 0.04% | 0.05% | |
| Myristic Acid | 0.44% | 0.26% | 0.19% | |
| Ethyl Myristate | 0.12% | 0.09% | | |
| Palmitic Alcohol | 0.00% | 0.00% | 0.03% | |
| Palmitic Acid | 9.22% | 7.86% | 12.69% | 12.42% |
| Ethyl Palmitate | 2.64% | 5.08% | 0.77% | |
| trans Phytol | 0.13% | 0.00% | | |
| Linoleic Acid/Oleic Acid | 34.24% | 35.13% | 69.77% | 71.52% |
| Ethyl Linoleate | 10.87% | 27.79% | 5.49% | 2.24% |
| Ethyl Oleate | 2.00% | | 7.20% | |
| Ethyl Stearate | 0.48% | 1.45% | | |
| Total identified | 100.00% | 100.00% | 100.00% | 100.00% |

TABLE 2

| | Raw Percentages | | | |
|---|---|---|---|---|
| | CABERNET | MERLOT | CHARDONNEY | REISLING |
| iso Propyl Alcohol | Trace | | | |
| Ethyl Acetate | 0.355 | | | |
| Acetic Acid | Trace | 0.041 | | 0.032 |
| iso Butanol | 0.502 | 0.382 | | |
| Ethyl Propionate | 0.097 | | | |
| Acetoin (3-Hydroxy-2-Butanone) | | 0.045 | | |
| iso Amyl Alcohol | 10.506 | 4.868 | 0.004 | |
| Ethyl Butyrate | 0.162 | | | |
| iso Valeric Acid | 0.038 | | | |
| Aldehyde C-6 | | Trace | 0.089 | 0.326 |
| Octene | | | | 0.172 |
| Octane | | | 0.316 | 0.866 |
| Leaf Alcohol (cis-3-Hexenol) | 0.008 | | | |
| Alcohol C-6 | 0.964 | | 0.037 | 0.049 |
| iso Amyl Acetate | 0.095 | 0.030 | | |
| Aldehyde C-7 | 0.016 | | 0.066 | 0.063 |
| Alcohol C-7 | | 1.524 | | |
| Caproic Acid | 0.144 | 0.183 | | 0.019 |
| 1-Octen-3-ol | 0.073 | | 0.040 | 0.175 |
| Ethyl Caproate | 1.095 | 0.183 | 0.022 | |
| Valeraldehyde Diethyl Acetal | | 0.010 | | |
| Hexyl Acetate | 0.079 | | | |
| Phenyl iso Butyrate | | | | 0.060 |
| Phenyl Acetaldehyde | 0.010 | | | |
| Alcohol C-8 | 0.038 | | | |
| Ethyl Heptoate | 0.006 | | | |
| Phenyl Ethyl Alcohol | 1.855 | 1.266 | 0.019 | |
| Diethyl Succinate | | 0.020 | | |
| Caprylic Acid | 0.423 | 0.298 | | |
| Ethyl Caprylate | 1.961 | 1.346 | 0.007 | |
| trans-2,trans-4-Decadienal | 1.270 | 0.861 | 1.057 | 2.738 |
| Decanoic Acid | 1.011 | 0.546 | | |
| Ethyl Decylate | 1.177 | 0.871 | | |
| iso Amyl Caprylate | 0.047 | | | |
| Lauric Acid | 0.860 | 0.459 | | |
| Ethyl Laurate | 0.231 | 0.213 | | |
| cis alpha Farnesol | 0.010 | | | |
| Benzyl Benzoate | | 0.023 | 0.020 | |
| Myristic Acid | 0.257 | 0.151 | 0.084 | |
| Ethyl Myristate | 0.067 | 0.051 | | |
| Palmitic Alcohol | | | 0.012 | |
| Palmitic Acid | 5.327 | 4.631 | 5.519 | 4.043 |
| Ethyl Palmitate | 1.523 | 2.991 | 0.335 | |
| trans Phytol | 0.076 | | | |
| Linoleic Acid/Oleic Acid | 19.785 | 20.697 | 30.341 | 23.284 |
| Ethyl Linoleate | 6.278 | 16.371 | 2.389 | 0.730 |
| Ethyl Oleate | 1.156 | | 3.132 | |
| Ethyl Stearate | 0.279 | 0.855 | | |
| Total identified (percent) | 57.781 | 58.916 | 43.489 | 32.557 |
| Total not identified (percent) | 42.219 | 41.084 | 56.511 | 67.443 |

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the particular method of obtaining the oils, the varietal grape seeds used, and oil blends which may be made. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. Unless otherwise indicated herein, all terms have their ordinary meanings as understood by one of ordinary skill in the field to which the invention pertains. Unless otherwise limited, the use of the article “a” or “an” is intended to include one or more. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

What is claimed is:

1. A modified edible oil comprising
a modified edible grape seed oil extracted from dried fermented grape seeds isolated from a fermented grape pomace.

2. The edible oil of claim 1, wherein said modified edible grape seed oil contains distinguishing flavor and fragrance chemicals evidenced by organoleptic evaluation or chemical analysis or both, wherein said chemicals are substantially lacking in grape seed oils that have been similarly extracted from grape seeds isolated from a non-fermented grape pomace.

3. The edible oil of claim 1, wherein said grape seed oil is physically extracted from said dried fermented grape seeds.

4. The edible oil of claim 1 wherein said oil is physically extracted and purified from grape seeds by cold-pressing without the use of organic solvent extraction of said oil.

5. The edible oil of claim 1, wherein said distinguishing flavor and fragrance chemicals comprise at least one organic alcohol.

6. The edible oil of claim 5, wherein said at least one organic alcohol is selected from the group consisting of isobutanol, isoamyl alcohol and phenylethyl alcohol.

7. The edible oil of claim 1 wherein said distinguishing flavor and fragrance chemicals comprise at least one organic ester.

8. The edible oil of claim 7, wherein said at least one organic ester is selected from the group consisting of ethyl propionate, ethyl butyrate, isoamyl acetate, ethyl caproate, hexyl acetate, ethyl caprylate, ethyl decylate, isoamyl caprylate, ethyl laurate and ethyl myristate.

9. The edible oil of claim 1 wherein said distinguishing flavor and fragrance chemicals comprise at least one organic acid.

10. The edible oil of claim 9, wherein said at least one organic acid is selected from the group consisting of acetic acid, isovaleric acid, caproic acid, caprylic acid, decanoic acid and lauric acid.

11. The edible oil of claim 1, wherein said fermented grape pomace is produced from at least one varietal grape of a grape species selected from the group consisting of Vitis vinifera (European wine grape), Vitis labrusca (Concord), and Vitis rotundifolia (Muscadine).

12. The edible oil of claim 11, wherein said fermented grape pomace is produced from the Merlot varietal grape variety of the species Vitis vinifera.

13. The edible oil of claim 11, wherein said fermented grape pomace is produced from the Cabernet Sauvignon varietal grape of the species Vitis vinifera.

14. The edible oil of claim 11, wherein said fermented grape pomace is produced from the grape species Vitis vinifera, wherein said one varietal grape of said species is selected from the group consisting of Barbarossa, Barbera, Cabernet Franc, Cabernet Sauvignon, Castiglione, Charbono, Corvina, Dolcetto, Dornfelder, Freisa, Gamay, Graciano, Gropello, Grignolino, Lambrusco, Malbec, Malvasia, Mandolari, Merlot, Millot, Mission, Molinara, Montepulciano, Negrette, Nerello, Petit Syrah, Petit Verdot, Pinot Noir, Pinotage, Poulsard, Rodinella, Rotberger, Rufete, Sagrantino, Sangiovese, Shiraz, Syrah, St. Laurent, Tarrango, Terret Noir, Touriga, Troia, Vranac, and Zinfandel.

15. The edible oil of claim 1, wherein said oil is used as an ingredient in a personal care or cosmetic product.

16. The edible oil of claim 15, wherein said personal care or cosmetic product is selected from the group consisting of massage oils, skin care oils, skin cleansers, skin moisturizers, skin serums, skin creams, body washes, body butters, body muds, body scrubs, bath gels, bath lotions, sun-blocking oils and sun lotions.

17. The edible oil of claim 1, further comprising at least one flavoring selected from the group consisting of smoke, citrus, herbal, savory, and pepper flavors.

18. An oil blend comprising the edible oil of claim 1 combined with a second edible triglyceride-based oil.

19. The oil blend of claim 18, comprising the edible oil of claim 2, wherein said distinguishing flavor and fragrance chemicals contribute to the flavor and fragrance of said oil blend.

20. The oil blend of claim 19, wherein said second edible oil is selected from the group consisting of raspberry seed oil, cranberry seed oil, and blueberry seed oil.

21. The oil blend of claim 19, wherein said second edible oil is selected from the group consisting of olive oil, soybean oil, canola oil, corn oil, peanut oil, palm oil, and flax seed oil.

22. A prepared food comprising the edible oil of claim 1.

23. The prepared food of claim 22, comprising the edible oil of claim 2, wherein said distinguishing flavor and fragrance chemicals contribute to the flavor and fragrance of said food product.

24. The prepared food of claim 23, wherein said prepared food is selected from the group consisting of salad dressings, dips, marinades, rubs, salsas, cooking sauces, and pasta sauces.
